(19) United States
(12) Patent Application Publication
(10) Pub. No.: US 2010/0129299 A1
(43) Pub. Date: May 27, 2010

(54) TOPICAL SUNSCREEN COMPOSITIONS FOR MITIGATING EFFECTS ON INTRACELLULAR ION CONCENTRATION

(76) Inventors: Laura C. Singleton, Los Angeles, CA (US); Theresa Chen, Hopewell, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/618,833

(22) Filed: Nov. 16, 2009

Related U.S. Application Data
(60) Provisional application No. 61/116,837, filed on Nov. 21, 2008.

(51) Int. Cl. A61K 8/49; A61Q 17/04

(52) U.S. Cl. 424/59

(57) ABSTRACT

The present invention relates to a composition suitable for topical application to skin to provide protection to the skin when exposed to UV radiation, the composition including a sunscreen in amounts effective to absorb, reflect or scatter radiation in at least a portion of the ultraviolet spectrum, a marine nucleotide in amounts effective to mitigate effects on intracellular ion concentration and a cosmetically-acceptable topical carrier.

This application claims the benefit of U.S. provisional application 61/116,837 filed on Nov. 21, 2008, the complete disclosure of which is hereby incorporated herein by reference for all purposes.

### FIELD OF THE INVENTION

The present invention relates to topical sunscreen compositions using a marine nucleotide to mitigate affects on intracellular ion concentration caused by exposure of skin to UV radiation.

### BACKGROUND OF THE INVENTION

It is known that skin is susceptible to damage from ultraviolet radiation. This known damage can be, for example, free radical damage, necrotic transformation and apoptosis. Conventional sunscreen compositions are formulated to absorb, deflect or scatter UV radiation utilizing known sunscreens, thus providing protection to the skin. Such sunscreens include organic sunscreen, including UV-A and UV-B absorbing moieties, as well as inorganic sunscreens, such as water insoluble particulate compounds that scatter UV radiation. However, such compositions are not necessarily known to address other affects to skin caused by exposure to UV radiation, particularly with respect to intracellular properties of the skin, for example intracellular ion concentration.

Therefore, there is a need to provide new sunscreen compositions that not only provide conventional protection of skin from exposure to UV radiation, but that also address other ill-effects caused by exposure of the skin to UV radiation. The present invention provides such compositions that synergistically provide conventional protection to the skin and address intracellular ion concentration imbalance caused by exposure to UV radiation.

### SUMMARY OF THE INVENTION

The present invention is directed to compositions suitable for topical application to the skin to provide protection from exposure of the skin to UV radiation. The compositions include a sunscreen in amounts effective to absorb, reflect or scatter radiation in at least a portion of the ultraviolet spectrum, a marine nucleotide in amounts effective to mitigate effects on intracellular ion concentration when the skin is exposed to UV radiation; and a cosmetically-acceptable topical carrier.

### DETAILED DESCRIPTION OF THE INVENTION

The inventors have surprisingly found that electrolyte imbalance can occur during UV exposure, particularly in skin cells. This affect may be exacerbated during sporting events or other activities resulting in perspiration during exposure of the skin to the UV radiation. In the case of heavy exudates caused by perspiring, an imbalance in ions can ensue, leading to more likelihood of damage, and the greater vulnerability of wet skin to UV damage. The inventors have further noted that in order to relieve, mitigate or prevent the potential damaging effects of electrolyte imbalance in skin cells, one can topically apply a composition comprising a marine nucleotide.

It is believed that one skilled in the art can, based upon the description herein, utilize the present invention to its fullest extent. The following specific embodiments are to be construed as merely illustrative, and not limiting of the remainder of the disclosure in any way whatsoever.

Unless defined otherwise, all technical and scientific terms used herein have the same meaning as commonly understood by one of ordinary skill in the art to which the invention belongs. Unless otherwise indicated, a percentage refers to a percentage by weight (i.e., % (W/W)).

### Definitions

As used herein, “topically applying” means directly laying on or spreading on outer skin, scalp, or hair, e.g., by use of the hands or an applicator such as a wipe, roller, or spray.

As used herein, “cosmetically-acceptable” means that the ingredients which the term describes are suitable for use in contact with tissues (e.g., the skin or hair) without undue toxicity, incompatibility, instability, irritation, allergic response, and the like.

As used herein, “safe and effective amount” means an amount of the electrolyte complex or of the composition sufficient to provide one or more benefits such as electrolyte replenishment, but low enough to avoid serious side effects. The safe and effective amount of the compounds or composition will vary with the area being treated, the age and skin type of the end user, the duration and nature of the treatment, the specific marine extract, ingredient, or composition employed, the particular cosmetically-acceptable carrier utilized, and other factors.

### Electrolyte Balancing Agent

Compositions of the present invention include an electrolyte balancing agent. By “electrolyte balancing agent” it is meant an ingredient that, when topically applied to skin cells or tissue, is capable of mitigating effects on intracellular ion concentrations caused by exposure of the skin to UV radiation. Such effects may include, for example but without limitation, an increase in intracellular ion concentration. Compositions of the present invention, in particular, include a marine extract, and particularly extremophiles such as halophiles and osmophiles or polyextremophiles. In one embodiment, the marine creature is capable of living in water having a salt concentration, such as higher than 3%, or even higher than 10% or 20%. These include creatures living in oceans, and especially salt lakes, or other high saline environments. Suitable marine life may include various animals, plants, monera, protists, algae and planktons, and preferably, Artemia salina.

“Artemia salina” is a species of brine shrimp or plankton, a primitive aquatic crustacean that can generally tolerate fluctuations in the salinity of water. They can live in water having much more or much less salt content than normal seawater.

By an “extract of Artemia salina,” it is meant a combination of compounds that are harvested from Artemia salina such as via biological, biochemical, or chemical extraction (e.g., via water or chemical solvents) techniques. The extract may include any of various proteins, carbohydrates, nutrients, excretion products and the like derived from Artemia salina. In one particularly preferred embodiment, the composition includes a marine protein or nucleotide, i.e., a nucleotide extracted from such a marine creature. In one particular embodiment, the marine nucleotide is diguanosine tetraphosphate.

The concentration of the Artemia salina extract in the inventive composition may be at least about 50 ppm, such as at least about 100 ppm, such as at least about 150 ppm (150 ppm is equivalent to 0.015% by weight). In another embodiment, the composition specifically has a concentration of marine nucleotide that is at least about 5 ppm, such as at least about 15 ppm. A notable form of Artemia salina extract is available from ISP/Vincience of Sophia Antiplos Cedex, France and sold as “Artemion.”

In another embodiment, the electrolyte balancing agent is a combination of ions. By “combination of ions,” it is meant a combination of at least three of the following ions: zinc, sodium, calcium, potassium, and magnesium, manganese, and selenium. In a preferred embodiment, the ions are chemically unbound (unlike in the case of an insoluble mineral such as zinc oxide, in which the zinc is bound to the oxide and essentially unavailable for interaction with the skin) As such, the ions are preferably either merely solvated (dissolved) by water in the composition, and readily available as ions to interact with skin to which the composition is applied. Although it is preferred that the ions be unbound, in one embodiment, the one or more (particularly zinc, calcium, manganese, selenium, and/or magnesium) ions may be present in sequestered or chelated form. In another embodiment, the ions may be present as bound salts or chelates in the composition, but when the composition is applied to the skin, the combination of ions are available in free ionic form (or alternatively as chelates) to interact with the skin.

In one embodiment, the combination of ions includes at least four of the following ions: zinc, sodium, calcium, potassium, manganese, selenium and magnesium. In one particularly notable embodiment, the combination of ions includes potassium ions, and 2 or more of calcium, manganese, selenium, magnesium, and zinc ions. The two or more of calcium, magnesium, manganese, selenium and zinc ions may be present in chelated form, as free ions, or as a combination of both of these states. In another embodiment, the combination of ions includes all of the following ions: zinc, sodium, calcium, and potassium ions.

The combination of ions may be present in the composition in a total concentration of about 1 ppm, or at least about 10 ppm, such as at least about 25 ppm, such as at least about 50 ppm. The ions may be derived from any of various salts that are suitable for use in a topically applied product. For example, the zinc, sodium, calcium, manganese, and selenium, and magnesium may be supplied to the composition via chloride slats, sulfate salts, or other soluble salt forms. The salts should have water solubility that is sufficient to provide a safe and effective amount of ions in the composition. In a preferred embodiment, the composition comprises (i) an extract of Artemia salina, (ii) yeast extract, and (iii) a combination of ions described above.

On certain embodiments, combination of ions may be in the form of a yeast extract comprising the combination of ions. The yeast extract may be an extract of genus Saccharomyces, e.g., Saccharomyces cerevisiae, Saccharomyces bayanus, used in or Saccharomyces boulardii. The yeast extract may be obtained by any suitable biological, biochemical, or chemical extraction (e.g., via water or chemical solvents) techniques. In one embodiment, the yeast extract is a yeast protein. The yeast extract may be present in a concentration of at least about 10 ppm, such as at least about 25 ppm, such as at least about 50 ppm.

### Sunscreen

Compositions of the present invention may include a sunscreen. The inventors have surprisingly found that marine extracts, particularly extracts of Artemia salina and/or marine nucleotides such as diguanosine tetraphosphate, greatly enhance the ability of sunscreens to protect skin cells from UV-induced adverse affects in cellular morphology and the like.

By “sunscreen” it is meant, a molecule that absorbs, reflects or scatters radiation in at least a portion of the ultraviolet spectrum. In one embodiment, the sunscreen includes an UV-A absorbing moiety. By “UV-A absorbing moiety,” it is meant that the moiety has appreciable absorbance in the UV-A portion (320 nm to 400 nm) of the ultraviolet spectrum. For example, when a compound that includes the particular chemical moiety is cast into a film, it is possible to generate a molar extinction coefficient measured for at least one wavelength in this wavelength range is at least about 1000 mol⁻¹ cm⁻¹, preferably at least about 2000 mol⁻¹ cm⁻¹, more preferably at least about 4000 mol⁻¹ cm⁻¹. In a preferred embodiment, the molar extinction coefficient among at least 40% of the wavelengths in this portion of the spectrum is at least about 1000 mol⁻¹ cm⁻¹.

Examples of moieties that are UV-A absorbing include tertrahydroxybenzophenones; dicarboxydihydroxybenzophenones and alkane ester or acid halide derivatives thereof; dihydroxy-, dicarboxy-, and hydroxycarboxydibenzoylmethanes and alkane ester or acid halide derivatives thereof; dihydroxy-, dicarboxy-, and hydroxycarboxystilbenes and alkane ester or acid halide derivatives thereof; bis(hydroxystyrenyl)benzenes; bis(carboxystyrenyl)benzenes and alkane ester or acid halide derivatives thereof; dihydroxy-, dicarboxy, and hydroxycarboxycarotenes and alkane ester or acid halide derivatives thereof; 2 cyano-3,3-diphenyl acrylic acid, 2-ethyl hexyl ester; and any suitably functionalized species capable of copolymerization within the polymer chain capable of absorbing ultraviolet light in the 320-400 nm range.

In one particularly preferred embodiment, the UV-absorbing moiety is a UV-absorbing triazole and/or a UV-absorbing benzoylmethane.

By “UV-absorbing triazole” it is meant UV-absorbing moiety containing a five-membered heterocyclic ring with two carbon and three nitrogen atoms. UV-absorbing triazoles include, for example, compounds of the formula (I) or (II):

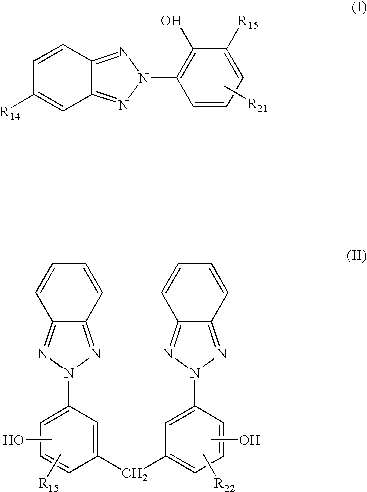

wherein R₁₄ is an optional C₁-C₁₈ alkyl or hydrogen; R₁₅ and R₂₂, independently, are optionally C₁-C₁₈ alkyl that may be substituted with a phenyl group, and R₂₁ is an optional C₁-C₈ alkyl. For (I), either of the R₁₄, R₁₅, or R₂₁ group may be oriented so as to be directly bonded to the (ester) linking group that connects the UV-absorbing dibenzoylmethane to the C—C backbone. For (II), either of the R₁₅ or R₂₂ group may be oriented so as to be directly bonded to the (ester) linking group that connects the UV-absorbing triazole to the C—C backbone.

Examples of compounds of Formulae (I) and (II) are described in U.S. Pat. No. 5,869,030, and include, but are not limited to, methylene bis-benzotriazolyl tetramethylbutylphenol (TINSORB M, Ciba Specialty Chemicals Corporation, Greensboro, N.C., USA). Other UV-absorbing dibenzoylmethanes include 2-(4-diethyl amino-2 hydroxybenzol)-benzoic acid hexylkester, commercially available as UVINUL A Plus from BASF of Parsippany, N.J.

UV-absorbing dibenzoylmethanes include those that may be represented by formula (III):

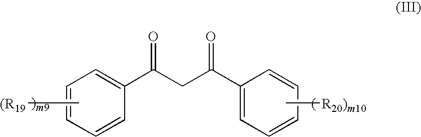

wherein R₁₉ and R₂₀, independently, are optional C₁-C₈ alkyl or C₁-C₈ alkoxy, m9 is 0 to 3, and m10 is 1 to 3.

Examples and the synthesis of such monomeric dibenzoylmethane compounds compositions are disclosed in U.S. Pat. No. 4,489,057 and include, but are not limited to, 4-(1,1-dimethylethyl)-4′-methoxydibenzoylmethane (avobenzone and sold as PARSOL 1789, Roche Vitamins and Fine Chemicals, Nutley, N.J., USA), 2-2-methyldibenzoylmethane, 4-methyl-dibenzoylmethane, 4-isopropyldibenzoylmethane, 4-tert-butyldibenzoylmethane, 4-tert-butyl-4′-methoxydibenzoylmethane, 2,4-dimethylbenzoylmethane, 2,5-dimethylbenzoylmethane, 4,4′-diisopropylbenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, and 2,6-dimethyl-4-tert-butyl-4′-methoxydibenzoylmethane.

In another embodiment, the sunscreen includes a UV-B absorbing moiety. By “UV-B absorbing moiety,” it is meant that the moiety has appreciable absorbance in the UV-B portion (290 nm to 320 nm) of the ultraviolet spectrum. In one embodiment, the criteria for consideration as a UV-B absorbing moiety is similar to those described above for an UV-A absorbing moiety, except that the wavelength range is 290 nm to 320 nm.

Examples of suitable UV-B absorbing moieties include 4-aminobenzoic acid and alkane esters thereof; anthranilic acid and alkane esters thereof; salicylic acid and alkane esters thereof; hydroxycinnamic acid alkane esters thereof; dihydroxy-, dicarboxy-, and hydroxycarboxybenzophenones and alkane ester or acid halide derivatives thereof; dihydroxy-, dicarboxy-, and hydroxycarboxychalcones and alkane ester or acid halide derivatives thereof; dihydroxy-, dicarboxy-, and hydroxycarboxycoumarins and alkane ester or acid halide derivatives thereof; and other suitably functionalized species capable of copolymerization within the polymer chain.

Particularly suitable UV-B absorbing moieties include UV-absorbing benzophenones and UV-absorbing diphenylcyanoacrylate derivatives. Examples of benzophenone derivatives include those known in the art to provide protection of the skin from UV radiation, for example, such as taught by U.S. Pat. No. 5,776,439. Preferred compounds include 2-hydroxy-4-methoxybenzophenone (“oxybenzone”) and 2-2′dihyroxy-4-methoxybenzophenone (“dioxybenzone”) and diethylamine hydroxybenzoyl hexyl benzoate (“hydroxybenzophenone”). Examples of diphenylcyanoacrylate derivatives include 2-ethylhexyl 2-cyano-3,3-diphenyl-2-propenoate (“octocrylene”).

While the sunscreens in the composition may be limited to “organic sunscreens” (also known as UV filters), typically those that absorb radiation in the UV, in certain embodiments, the composition may also include “physical” sunscreens, generally water insoluble particulate compounds that scatter UV radiation. Examples of such physical sunscreens include zinc oxide and titanium oxide.

The concentration of sunscreen(s) used in compositions of the present invention will depend upon such factors as the selection and/or combination of sunscreens, the level of protection desired and the formulation of the particular composition. Compositions according to the invention typically may comprise from about 2% to about 50% by weight of the sunscreen, or from about 5 to about 40%, of from about 10 to about 30%.

### Waterproofing Polymer

In one embodiment of the invention, the composition includes one or more waterproofing polymers. By “waterproofing polymers” it is meant any of those polymers suitable for binding sunscreens to the skin.

Examples of waterproofing polymers include, but are not limited to, PVP, PVP/Eicosene copolymer, tricontanyl PVP, PVP/hexadecene copolymer, hydrogenated polyisobutene, adipic acid/diethylene glycol/glycerin crosspolymer, octadecene/MA copolymer, synthetic wax, diglycol/CHDM/Isophthalates/SIP copolymer, silicon modified hydrolyzed proteins (Seiwa Kasei Co., Ltd. Of Osaka, Japan; PROMOIS SIG series). The one or more waterproofing polymers may be present in concentrations from about 0.1 to about 6%, preferably from about 2.5% to about 4.5%.

In one particular embodiment, the waterproofing polymer includes one or more of the following waterproofing polymers: (i) a silicon-modified acrylate (e.g., KP-545 from Shin-Etsu of Akron, OH), (ii) a wax, such as a natural wax, and preferably beeswax (e.g., cara alba; SP422P); (iii) a copolymer of maleic anhydride and long chain alpha-olefins such as an octadecene/maleic anhydride copolymer (e.g., PA-18 from Chevron), and (iv) a glycerin crosspolymer such as an adipic acid & diethylene glycol/glycerin crosspolymer (e.g., Lexorez 100 from Inolex), and mixtures thereof.

### Topical Compositions

The topical compositions useful in the present invention are suitable for topical application to the skin. In one embodiment, the composition contains (i) a safe and effective amount of an electrolyte complex, (ii) a sunscreen, and (iii) a cosmetically-acceptable topical carrier. In one embodiment, the cosmetically-acceptable topical carrier is from about 50% to abut 99.99%, by weight, of the composition (e.g., from about 80% to about 99%, by weight, of the composition. In a preferred embodiment of the invention, the cosmetically-acceptable topical carrier includes or consists essentially of water.

The compositions may be made into a wide variety of product types that include but are not limited to lotions, creams, gels, sticks, sprays, ointments, cleansing liquid washes and solid bars, shampoos and hair conditioners, hair fixers, pastes, foams, powders, mousses, shaving creams, wipes, patches, nail lacquers, wound dressing and adhesive bandages, hydrogels, facial masks and skin masks, films and make-up such as foundations, mascaras, and lipsticks. These product types may contain several types of cosmetically-acceptable topical carriers including, but not limited to solutions, suspensions, emulsions such as microemulsions and nanoemulsions, gels, solids and liposomes. The following are non-limitative examples of such carriers. Other carriers can be formulated by those of ordinary skill in the art.

The topical compositions useful in the present invention can be formulated as solutions. While it preferred that the solution be aqueous, in certain embodiments the composition may, in addition to, or in place of water, include cosmetically acceptable organic solvents. Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, and mixtures thereof. The one or more solvents may be present from about 50% to about 99.99% or from about 90% to about 99% of a cosmetically acceptable aqueous or organic solvent.

Topical compositions useful in the subject invention may be formulated as a solution comprising an emollient. Such compositions preferably contain from about 2% to about 50% of an emollient(s). As used herein, “emollients” refer to materials used for the prevention or relief of dryness, as well as for the protection of the skin or hair. Examples of emollients include, but are not limited to, those set forth in the International Cosmetic Ingredient Dictionary and Handbook, eds. Pepe, Wenninger and McEwen, pp. 2930-36 (The Cosmetic, Toiletry, and Fragrance Assoc., Washington, D.C., 9^{th} Edition, 2002) (hereinafter “ICI Handbook”).

A lotion can be made from such a solution. Lotions typically contain from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Although it is preferred that the topical composition of the present invention includes water, the topical compositions may alternatively be anhydrous compositions or ointments that include no water but organic and/or silicone solvents, oils, lipids and waxes. An ointment may contain a simple base of animal or vegetable oils or semi-solid hydrocarbons. An ointment may contain from about 2% to about 10% of an emollient(s) plus from about 0.1% to about 2% of a thickening agent(s). Examples of thickening agents include, but are not limited to, those set forth in the ICI Handbook pp. 2979-84.

The topical compositions useful in the present invention formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 2962-71.

Lotions and creams can be formulated as emulsions. Typically such lotions contain from 0.5% to about 5% of an emulsifier(s). Such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the cosmetic art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The topical compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The topical compositions of the present invention can also be formulated into a solid formulation (e.g., a wax-based stick, soap bar composition, powder, or a wipe containing powder).

The topical compositions useful in the subject invention may contain, in addition to the aforementioned components, a wide variety of additional oil-soluble materials and/or water-soluble materials conventionally used in compositions for use on skin, hair, and nails at their art-established levels.

### Additional Cosmetically-Active Agents

In one embodiment, the topical composition further contains another cosmetically active agent in addition to the phospholipids having a single fatty-acid moiety. What is meant by a “cosmetically-active agent” is a compound (e.g., a synthetic compound or a compound isolated from a natural source) that has a cosmetic or therapeutic effect on the skin, hair, or nails, including, but not limiting to, anti-acne agents, shine control agents, anti-microbial agents, anti-inflammatory agents, anti-mycotic agents, anti-parasite agents, external analgesics, sunscreens, photoprotectors, antioxidants, keratolytic agents, detergents/surfactants, moisturizers, nutrients, vitamins, energy enhancers, astringents, deodorants, hair removers, firming agents, anti-callous agents, and agents for hair, nail, and/or skin conditioning.

### Other Materials

Various other materials may also be present in the compositions useful in the subject invention. These include humectants, pH adjusters, minerals, and preservatives (e.g., parabens). Examples of such agents are listed in pp. 2922-23, 2926-28, and 2892 of the ICI Handbook. In addition, the topical compositions useful herein can contain conventional cosmetic adjuvants, such as dyes, opacifiers (e.g., titanium dioxide), and fragrances.

Compositions of the present invention may be prepared using methodology that is well known by an artisan of ordinary skill. Specific examples of inventive polymers, methods, and compositions are described below.

### Inventive Example 1

| TRADE NAME/ | | |
|---|---|---|
| SUPPLIER | INGREDIENT | Wt. % |
| Water Phase | Water | 48.97 |
| Pemulen TR1/Noveon | Acrylates/C10 30 Alkyl Acrylate | 0.500 |
| | Crosspolymer | |
| Brenntag West Co. | Disodium EDTA | 0.100 |
| OrinetStarr Co | Dipotassium Glycyrrhizate | 0.100 |
| Clariant Co | Methylparaben | 0.350 |
| D&D Chem Co. | Ethylparaben | 0.250 |
| Oil Phase | | |
| Homosalate/Symrise | Homosalate | 15.000 |
| Octyl | Ethylhexyl Salicylate | 5.000 |
| Salicylate/Symrise | | |
| Avobenzone/Symrise | Avobenzone | 3.000 |
| Oxybenzone/Symrise | Benzophenone - 3 | 5.000 |
| Octocrylene/Symrise | Octocrylene | 4.000 |
| Hallbrite TQ/Symrise | Diethylhexyl 2,6-Naphthalate | 0.100 |
| Sensiva/Shulke&mayer | Ethylhexylglycerin | 0.500 |
| Arlacel165 | Glyceryl Stearate & PEG-100 | 1.600 |
| VEG/Univar | Stearate | |
| UnivarUSA | BHT | 0.070 |
| SP422P/Lipscomb | Beeswax | 0.750 |
| KP-545/CShinEtsu | Cyclopentasiloxane Acrylates | 0.500 |
| | Dimethicone Copolymer | |
| PA-18/Chevron | Octadecene/MA Copolymer | 1.000 |
| Lexorez100/Inolex | Adipic Acid & Diethylene Glycol | 2.000 |
| | & Glycerin Crosspolymer | |
| Clariant | Propylparaben | 0.200 |
| Neutralization Phase | Water | 5.000 |
| Triethanolamine 99%/ | Triethanolamine | 0.650 |
| UnivarUSA | | |
| Post Adds | | |
| Silispheres 10M/Argan | Silica | 3.000 |
| Charkit Chem. | Benzyl Alcohol | 1.000 |
| Neolone 950/Rhom | Methylisothiazolinone | 0.100 |
| &Has | | |
| Alpha-Bisabolol, | Bisabolol | 0.100 |
| Natural/Symrise | | |
| Artemion ISP-Vincience | Water & Glycerin & Artemia Salina | 1.000 |
| | Extract | |
| NTG Electrolyte | Water & Saccharomyces Zinc | 0.010 |
| Complex/Active | Ferment & Saccharomyces | |
| Concepts | Magnesium Ferment & | |
| | Saccharomyces Sodium Ferment & | |
| | Saccharomyces Calcium Ferment & | |
| | Saccharomyces Potassium Ferment | |
| Pleasure Island 404421 | Frag. Firmenich, Pleasure Island | 0.150 |
| D/Firmenich | 404421 D | |
| | | 100.00 |

### Comparative Example 2

| TRADE NAME/ | | |
|---|---|---|
| SUPPLIER | INGREDIENT | Wt. % |
| Water Phase | Water | 81.98 |
| Pemulen TR1/Noveon | Acrylates/C10 30 Alkyl Acrylate | 0.500 |
| | Crosspolymer | |
| | Disodium EDTA | 0.100 |
| OrinetStarr | Dipotassium Glycyrrhizate | 0.100 |
| | Methylparaben | 0.350 |
| | Ethylparaben | 0.250 |
| Oil Phase | | |
| Homosalate/Symrise | Homosalate | 0.000 |
| Octyl | Ethylhexyl Salicylate | 0.000 |
| Salicylate/Symrise | | |
| Avobenzone/Symrise | Avobenzone | 0.000 |
| Oxybenzone/Symrise | Benzophenone - 3 | 0.000 |
| Octocrylene/Symrise | Octocrylene | 0.000 |
| Hallbrite TQ/Symrise | Diethylhexyl 2,6-Naphthalate | 0.100 |
| Sensiva/Shulke&mayer | Ethylhexylglycerin | 0.500 |
| Arlacel165 | Glyceryl Stearate & PEG-100 | 1.600 |
| VEG/Univar | Stearate | |
| UnivarUSA | BHT | 0.070 |
| SP422P/Lipscomb | Beeswax | 0.750 |
| KP-545/CShinEtsu | Cyclopentasiloxane Acrylates | 0.500 |
| | Dimethicone Copolymer | |
| PA-18/Chevron | Octadecene/MA Copolymer | 1.000 |
| Lexorez100/Inolex | Adipic Acid & Diethylene Glycol | 2.000 |
| | & Glycerin Crosspolymer | |
| | Propylparaben | 0.200 |
| Neuralization Phase | Water | 5.000 |
| Triethanolamine 99%/ | Triethanolamine | 0.650 |
| UnivarUSA | | |
| Post Adds | | |
| Silispheres 10M/Argan | Silica | 3.000 |
| Charkit Chem. | Benzyl Alcohol | 1.000 |
| Neolone 950/Rhom | Methylisothiazolinone | 0.100 |
| &Has | | |
| Alpha-Bisabolol, | Bisabolol | 0.100 |
| Natural/Symrise | | |
| Artemion ISP-Vincience | Water & Glycerin & Artemia Salina | 0.000 |
| | Extract | |
| NTG Electrolyte | Water & Saccharomyces Zinc | 0.000 |
| Complex/Active | Ferment & Saccharomyces | |
| Concepts | Magnesium Ferment & | |
| | Saccharomyces Sodium Ferment & | |
| | Saccharomyces Calcium Ferment & | |
| | Saccharomyces Potassium Ferment | |
| Pleasure Island 404421 | Frag. Firmenich, Pleasure Island | 0.150 |
| D/Firmenich | 404421 D | |
| | | 100.00 |

### Comparative Example 3

| TRADE NAME/ | | |
|---|---|---|
| SUPPLIER | INGREDIENT | Wt. % |
| Water Phase | Water | 49.98 |
| Pemulen TR1/Noveon | Acrylates/C10 30 Alkyl Acrylate | 0.500 |
| | Crosspolymer | |
| | Disodium EDTA | 0.100 |
| OrinetStarr | Dipotassium Glycyrrhizate | 0.100 |
| Clariant | Methylparaben | 0.350 |
| D&D Chem | Ethylparaben | 0.250 |
| Oil Phase | | |
| Homosalate/Symrise | Homosalate | 15.000 |
| Octyl | Ethylhexyl Salicylate | 5.000 |
| Salicylate/Symrise | | |
| Avobenzone/Symrise | Avobenzone | 3.000 |
| Oxybenzone/Symrise | Benzophenone - 3 | 5.000 |
| Octocrylene/Symrise | Octocrylene | 4.000 |
| Hallbrite TQ/Symrise | Diethylhexyl 2,6-Naphthalate | 0.100 |
| Sensiva/Shulke&mayer | Ethylhexylglycerin | 0.500 |
| Arlacel165 | Glyceryl Stearate & PEG-100 | 1.600 |
| VEG/Univar | Stearate | |
| UnivarUSA | BHT | 0.070 |
| SP422P/Lipscomb | Beeswax | 0.750 |
| KP-545/CShinEtsu | Cyclopentasiloxane Acrylates | 0.500 |
| | Dimethicone Copolymer | |
| PA-18/Chevron | Octadecene/MA Copolymer | 1.000 |
| Lexorez100/Inolex | Adipic Acid & Diethylene Glycol | 2.000 |
| | & Glycerin Crosspolymer | |
| Clariant | Propylparaben | 0.200 |
| Neutralization Phase | Water | 5.000 |
| Triethanolamine 99%/ | Triethanolamine | 0.650 |
| UnivarUSA | | |
| Post Adds | | |
| Silispheres 10M/Argan | Silica | 3.000 |
| Charkit Chem. | Benzyl Alcohol | 1.000 |
| Neolone 950/Rhom | Methylisothiazolinone | 0.100 |
| &Has | | |
| Alpha-Bisabolol, | Bisabolol | 0.100 |
| Natural/Symrise | | |
| Artemion ISP-Vincience | Water & Glycerin & Artemia Salina | 0.000 |
| | Extract | |
| NTG Electrolyte | Water & Saccharomyces Zinc | 0.000 |
| Complex/Active | Ferment & Saccharomyces | |
| Concepts | Magnesium Ferment & | |
| | Saccharomyces Sodium Ferment & | |
| | Saccharomyces Calcium Ferment & | |
| | Saccharomyces Potassium Ferment | |
| Pleasure Island 404421 | Frag. Firmenich, Pleasure Island | 0.150 |
| D/Firmenich | 404421 D | |
| | | 100.00 |

In the formulations above: glyceryl stearate and PEG-100 stearate are emulsifiers; Pemulen is a thickener/emulsifier; homosalate, ethylhexulsalicylate, avobenzone, benzophenone-3 and octocrylene are sunscreens; beeswax, Octadecene/MA Copolymerl, Adipic Acid & Diethylene Glycol & Glycerin Crosspolymer, and Cyclopentasiloxane Acrylates Dimethicone Copolymer are waterproofing polymers; BHT is an antioxidant; EDTA is a chelating agent; Dipotassium Glycyrrhizate and Bisabolol are anti-irritants; Ethylparaben, Propylparaben, Benzyl Alcohol and Methylisothiazolinone are preservatives; silica is an oil-absorbing powder. The pH was between 5 and 6.

NTG Electrolyte Complex/Active Concepts is a blend of water, Saccharomyces (yeast), zinc, magnesium, calcium, and potassium ions. Artemion is a blend of 1.5% Artemia salina extract in 30% glycerol and 68.5% water.

The above compositions may be made by processes known to those skilled in the art. For example, for the above compositions, in the primary container, the water phase ingredients are combined in a separate container: Water, Acrylates/C10 30 Alkyl Acrylate Crosspolymer, Disodium EDTA, Dipotassium Glyccyrrhizate, Methylparaben, Ethylparaben. Mix until acrylate the thickener is completely hydrated and heat the phase to 75-85 C.

The oil phase ingredients were then combined in a separate container: the 5 sunsccreens such as Homosalate, Ethylhexyl Salicylate, Avobenzone, Benzophenone-3, and Octocrylene, emollients such as Diethylhexyl 2,6-Naphthalate and Ethylhexylglycerin; anti-oxidant such as BHT, film formers such as Beeswax, Cyclopentasiloxane and Acrylates/Dimethicone Copolymer, Octadecene/MA Copolymer and Adipic Acid/Diethylene Glycol/Glycerin Crosspolymer. Mix until homogeneous and heat the phase to 75-85 C.

Emulsification: while mixing the water phase add slowly the oil phase with high mixing speed, and mixed until the emulsion is well formed. After the emulsion is formed it was homogenized for 2 min at 70% power.

The composition is then cooled and with continuous mixing and Silica powder was added, followed by the rest of the “post-add” ingredients. Mixing was then continued until emulsion is homogenous.

### Evaluation of Artemia Salina (Brine Shrimp) Extract for Affect on Intracellular Sodium and Chloride Concentrations

Normal human keratinocytes were cultured and incubated for fluorescence microscopy and quantification. The cells were incubated for 24 hours in a cell culture medium either with or without 1% “Artemion” (brine shrimp extract from ISP/Vincience) in the culture medium. Since Artemion is 1.5% Artemia salina extract, the concentration of Artemia salina extract used was 150 ppm. Each set of keratinocytes was separately irradiated with a very high dose of UVB irradiation (50 mJ/cm²).

Sodium was labeled with CoroNa™ Green Indicator. Sodium was detected via fluorescence detection microscopy and quantified by a microplate reader. For the cells treated with the control solution (no Artemion), the UV radiation greatly affected the concentration of intracellular sodium. Specifically, intracellular sodium increased by nearly 15%. Surprisingly for the Artemion treated cells, the intracellular sodium remained essentially constant, despite the high UV dose exposure.

These results are surprising firstly in that UV-radiation significantly affects intracellular sodium ion concentration and secondly, that the affect can be mitigated by Artemia salina extract.

### Evaluation of Compositions Including Sunscreen and Artemia Salina Extract for Effects of U-V-Induced Changes In Cell Morphology

Skin “explant” samples were obtained from the lower arms of human females. The samples were then formed into 6 mm punch biopsies. The 6 mm skin samples were either intentionally moisturized with phosphate buffer solution or left unmoisturized. The samples were then treated by spreading 20 microliters of various test solutions on the samples and allowing them to remain for 24 hours. The test samples separately evaluated were: Inventive Example 1 and Comparative Examples 2 and 3. A positive control was also performed in which the skin sample were not contacted with any test material, and not treated with UV. A negative control was performed by not contacting the skin sample with any test material, but still irradiating it with UV.

The skin samples were then irradiated with 100 mJ/cm2 of UV-B radiation. H&E (standard histology) staining was performed on the skin samples.

A dermatologist/dermapathologist grader examined the images of the stained cells. Based upon examining the epidermal junction and the counting the concentration of sunburn cells (noting cell “vacuoles” as an indication of cell membrane disruption), the grader concluded that Inventive Example 1 and Comparative Examples 2 and 3 each showed better structure and morphology than the negative control.

In particular, the grader determined the following concentrations of sunburn cells per unit length in the samples under unmoisturized (dry) and moisturized (wet) conditions:

| Test Material | Dry Conditions | Wet Conditions |
|---|---|---|
| None; no UV-irradiation | 0.007 | 0.046 |
| (Positive Control) | | |
| Inventive Example 1 | 0.023 | 0.077 |
| Comparative Example 2 | 0.216 | 0.206 |
| Comparative Example 3 | 0.129 | 0.190 |
| None (Negative Control) | 0.214 | 0.334 |

Comparative decrease in sunburn cell density was calculated in order to measure the decrease in protection performance of Comparative Example 3 (Sunscreen) vs. Inventive Example 1 (Sunscreen+Artemion extract). For dry conditions this was calculated as 100×(0.129−0.023)/0.129=82%. Similarly for wet conditions, this was calculated as 59%.

The raw data and above calculations surprisingly suggests that by including Artemia salina, the ability of composition with sunscreen to protect cells from UV-radiation-induced damage is significantly improved. It also surprisingly suggests that compositions of the present invention even provide these marked advantages even in wet environments, when skin is generally more susceptible to UV-damage.

We claim:

1. A topical composition suitable for topical application to the skin to provide protection from exposure of the skin to UV radiation, comprising:
a sunscreen in amounts effective to absorb, reflect or scatter radiation in at least a portion of the ultraviolet spectrum,
a marine nucleotide in amounts effective to mitigate effects on intracellular ion concentration when the skin is exposed to UV radiation; and
a cosmetically-acceptable topical carrier.

2. The topical composition of claim 1 comprising at least 5 ppm of said marine nucleotide.

3. The topical composition of claim 2 wherein said marine nucleotide comprises an extract of Artemia Salina.

4. The topical composition of claim 3 comprising at least 15 ppm of said Artemia Salina extract.

5. The topical composition of claim 3 comprising at least 100 ppm of said Artemia Salina extract.

6. The topical composition of claim 2 wherein said marine nucleotide comprises diguanosine tetraphosphate.

7. The topical composition of claim 6 comprising at least 15 ppm of said diguanosine tetraphosphate.

8. The topical composition of claim 6 comprising at least 100 ppm of said diguanosine tetraphosphate.

9. The topical composition of claim 2 comprising from about 2% to about 50% by weight of said sunscreen.

10. The topical composition of claim 4 comprising from about 5% to about 40% by weight of said sunscreen.

11. The topical composition of claim 5 comprising from about 5% to about 40% by weight of said sunscreen.

12. The topical composition of claim 7 comprising from about 5% to about 40% by weight of said sunscreen.

13. The topical composition of claim 8 comprising from about 5% to about 40% by weight of said sunscreen.

14. The topical composition of claim 1 wherein said sunscreen is selected from the group consisting of a UV-A absorbing moiety, a UV-B absorbing moiety and water insoluble particulate compounds that scatter UV radiation.

15. The composition of claim 1 further comprising a combination of at least three ions selected from the group consisting of zinc, sodium, calcium, potassium, and magnesium, manganese and selenium.

16. The composition of claim 15 wherein said combination of ions is present in said composition in the form of a yeast extract.

17. The composition of claim 1 further comprising a waterproofing polymer.
